(19) United States
(12) Patent Application Publication
(10) Pub. No.: US 2014/0100619 A1
(43) Pub. Date: Apr. 10, 2014

(54) ANTERIOR SPINE ARRAY CLAMP

(71) Applicant: Christian P. DiPaola, Shrewsbury, MA (US)

(72) Inventor: Christian P. DiPaola, Shrewsbury, MA (US)

(73) Assignee: University of Massachusetts, Shrewsbury, MA (US)

(21) Appl. No.: 13/748,520

(22) Filed: Jan. 23, 2013

Related U.S. Application Data
(60) Provisional application No. 61/589,859, filed on Jan. 23, 2012.

(51) Int. Cl. A61B 19/00

(52) U.S. Cl. CPC A61B 19/26
USPC 606/86 R

(57) ABSTRACT

An apparatus that connects an imaging treatment guidance system (ITGS) to a fixed location in a patient. One example of an ITGS is the Medtronic StealthStation® Treatment Guidance System used in the O-arm™ Surgical Imaging System. The apparatus is fixed relative to a patient at a proximal end, and supports the ITGS at a distal end. The apparatus has a plurality of adjustably-joined arms. In one embodiment, the proximal end of the apparatus is attached to a pin distractor fixed to the anterior spine of a patient. The apparatus provides multiplanar adjustability of the array in space. The apparatus can be designed such that it is compatible with many navigation systems by making the connection at a distal end a universal type clamp. In another embodiment a fixed arm is provided in which the joints or hinges are eliminated.

### CROSS-REFERENCE TO RELATED APPLICATIONS

This application claims priority to and the benefit of co-pending U.S. provisional patent application Ser. No. 61/589,859, filed Jan. 23, 2013, which application is incorporated herein by reference in its entirety.

### FIELD OF THE INVENTION

The invention relates to medical instruments in general and particularly to an instrument used in making medical images.

### BACKGROUND OF THE INVENTION

Various apparatus and methods are available for observing a field in which surgery is to be performed. Examples relating to cranial surgery and spinal surgery are now described.

The paper by Barazi S A, El-Maghraby H, Selway R, Marsh H., “Awake craniotomy using stealth frameless stereotaxy without rigid skull fixation,” Br J Neurosurg. 2005 February;20(1):43-5, describes a technique using the Medtronic Stealth spinal reference array that allows awake craniotomy to be performed without cranial fixation in the Mayfield pin head rest. A Medtronic spinal reference array (four-point H-shaped LED array) is fitted to a Yasargil footplate via a three-jointed swingarm. The Yasargil footplate is directly attached to the cranium after craniotomy and following stereotactic registration the patient is awakened. The patient is free to move his head during the procedure as the reference array does not move in relation to the cranial contents and the fiducials, preserving accuracy.

The paper by Francesco Costa et al., “Evaluation of the rate of decompression in anterior cervical corpectomy using an intra-operative computerized tomography scan (O-Arm system),” Eur Spine J DOI 10.1007/s00586-011-2028-7, 24 Sep. 2011, describes an evaluation of the efficacy of intra-operative computerized tomography (CT) scanning in the analysis of bone removal accuracy during anterior cervical corpectomy, in order to allow any necessary immediate correction in the event of inadequate bone removal.

### Medtronic Stealth Spinal Reference Array

Medical imaging systems are known in the prior art. By way of example, the StealthStation® treatment guidance system, from Medtronic Surgical Navigation Technologies (SNT) of 710 Medtronic Parkway, Minneapolis, Minn. 55432-5604 USA, is an accepted image-guided surgery system in the world, with more than 650 of the systems in use around the world as of July 2001, according to Medtronic SNT.

The StealthStation® treatment guidance system provides surgeons with a way to navigate through the body using 3D images as their guide. Images from a variety of traditional imaging sources including X-ray, computerized tomography (CT) scan, magnetic resonance imaging (MRI) and ultrasound are taken and are transformed into 3D images. Those images can be manipulated to provide a view of a location in a patient. The images can be used both before the operation takes place as well as in the operating room. The images can be used to assist a surgeon to focus on a location to be reached during surgery, without compromising nearby muscle, tissue, nerves or blood vessels.

A newer system is the StealthStation® 57® from Medtronic SNT. According to Medtronic, the StealthStation S7 interfaces seamlessly with multiple intraoperative imaging systems, including iMRI, iCT, C-arms, and Medtronic's O-arm® 3D imaging and 2D fluoroscopy system.

According to Medtronic, the O-arm® Surgical Imaging System seamlessly interfaces with the StealthStation® Navigation Systems to provide surgeons with real-time information about the patient's anatomy.

There is a need for a system and a method for providing fixation of a navigation array adjacent to, or in conjunction with anterior spine surgery.

### SUMMARY OF THE INVENTION

According to one aspect, the invention features an anterior spine array clamp. The anterior spine array clamp comprises an attachment clamp configured to mate removably with and configured to be fixed to a fixed location relative to an intended surgical site in a body of a patient, the attachment clamp representing a proximal end of the anterior spine array clamp; a first rigid arm having a first end adjustably attached to the attachment clamp and having a second end, and having a rotation axis disposed along the length of the first arm; a rotational connector adjustably attached to the second end of the first arm; a second rigid arm having a first end adjustably attached to the rotational connector and having a second end, and having a rotation axis disposed along the length of the second arm; and an array mating surface adjustably attached to the second end of the second arm, the array mating surface representing a distal end of the anterior spine array clamp, the array mating surface configured to allow a navigation array to be attached thereto; each of the attachment clamp, the first rigid arm, the rotational connector, the second rigid arm and the array mating surface provided with tightening screws configured to be operated manually, and each of said attachment clamp, said first rigid arm, said rotational connector, said second rigid arm and said array mating surface configured to be locked in place by tightening a respective tightening screw for a time period during a surgical operation.

In one embodiment, the fixed location relative to an intended surgical site in a body of a patient is a location in or on a mechanical device attached to the patient.

In another embodiment, the fixed location relative to an intended surgical site in a body of a patient is a location in or on a Caspar-type pin distractor attached to the patient.

In yet another embodiment, the anterior spine array clamp is constructed from a material that can be sterilized repeatedly.

In still another embodiment, the material is selected from the group consisting of titanium, aluminum and steel, alone or in combination.

According to another aspect, the invention relates to a method of using an anterior spine array clamp. The method comprises the steps of providing an anterior spine array clamp; attaching a structure to a location proximal to the intended surgical site in a body of a patient; removably attaching the attachment clamp of the anterior spine array clamp to the structure; and adjusting and locking in place the attachment clamp, the first rigid arm, the rotational connector, the second rigid arm and the array mating surface in relative positions. The anterior spine array clamp comprises an attachment clamp configured to mate removably with and configured to be fixed to a fixed location relative to an intended surgical site in a body of a patient, the attachment clamp representing a proximal end of the anterior spine array clamp; a first rigid arm having a first end adjustably attached to the attachment clamp and having a second end, and having a rotation axis disposed along the length of the first arm; a rotational connector adjustably attached to the second end of the first arm; a second rigid arm having a first end adjustably attached to the rotational connector and having a second end, and having a rotation axis disposed along the length of the second arm; and an array mating surface adjustably attached to the second end of the second arm, the array mating surface representing a distal end of the anterior spine array clamp, the array mating surface configured to allow a navigation array to be attached thereto; each of the attachment clamp, the first rigid arm, the rotational connector, the second rigid arm and the array mating surface provided with tightening screws configured to be operated manually, and each of said attachment clamp, said first rigid arm, said rotational connector, said second rigid arm and said array mating surface configured to be locked in place by tightening a respective tightening screw for a time period during a surgical operation.

In one embodiment, the method of using an anterior spine array clamp further comprises the step of attaching a navigation array to the array mating surface of the anterior spine array clamp.

In another embodiment, the method of using an anterior spine array clamp further comprises the step of adjusting a position of any of the attachment clamp, the first rigid arm, the rotational connector, the second rigid arm, the array mating surface and the navigation array relative to any other of the attachment clamp, the first rigid arm, the rotational connector, the second rigid arm, the array mating surface and the navigation array.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is an image of a prior art Stealthstation® Spine Referencing Set (item 9734495, Parts Catalog Medtronic Navigation 2010, page 18). In FIG. 1, clamps 105 and 110 have a structure at a proximal end that allows clamping to a bone in the spine of a patient and at a distal end a structure allowing a reference frame to be attached. The clamps 105 and 110 appear to have no articulation or rotation capability within the clamp itself. Reference frames 115, 120 and 125 are also shown. The reference frames can be used to hold imaging hardware such as digital camera hardware in a fixed location relative to the spine of the patient.

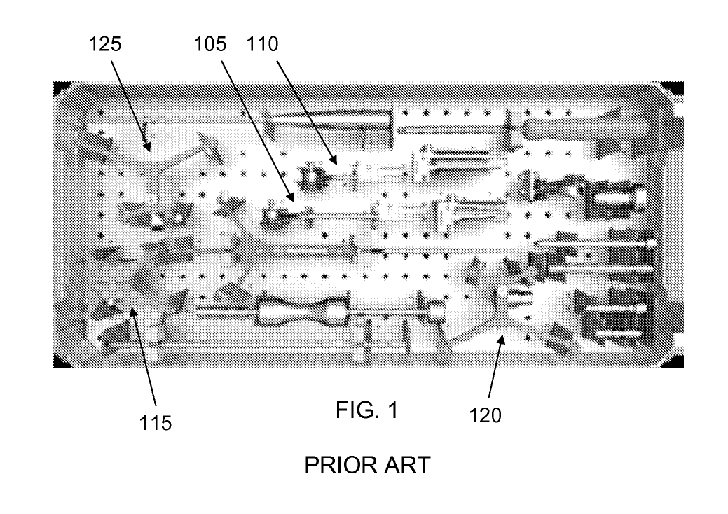

FIG. 2 is a diagram illustrating the anterior spine array clamp of the invention.

FIG. 3 is a diagram illustrating how the anterior spine array clamp is attached to a location in the anterior spine of a patent using a Caspar-type pin distractor.

### DETAILED DESCRIPTION

The anterior spine array clamp of the invention has as a purpose the fixing of an image guided navigation array solidly to the anterior spine (cervical, thoracic or lumbar). The anterior spine array clamp of the invention expands the usage of current image guided spinal navigations systems (such as but not limited to O-arm™/Stealth) to be available for use in surgery of the anterior spine.

The anterior spine array clamp provides a safer and more sterile system and method to apply the navigation arrays in the anterior spine. The systems employed heretofore cannot fix an array to the anterior spine with a sterile instrument. In addition, this system allows a more rigid connection of the fixed array in the navigation system to the anterior spine. It allows connection of a navigation array to the anterior spine as close as possible to the surgical field without obstructing the working area.

The anterior spine array clamp apparatus connects an imaging treatment guidance system (ITGS) to a fixed location in a patient. One example of an ITGS is the Medtronic StealthStation® Treatment Guidance System used in the O-arm™ Surgical Imaging System. The apparatus is fixed relative to a patient at a proximal end, and supports the ITGS at a distal end. The apparatus has a plurality of adjustably-joined arms. In one embodiment, the proximal end of the apparatus is attached to a Capar pin distractor fixed to the anterior spine of a patient. The apparatus provides multiplanar adjustability of the array in space. This can be accomplished with any number or variety of joints or hinges or could exist without any of the adjusting joints if a surgeon desires a fixed arm. The apparatus can be designed such that it is compatible with many navigation systems by making the connection at a distal end a universal type clamp. In another embodiment a fixed arm is provided in which the joints or hinges are eliminated.

The anterior spine array clamp apparatus could in principle be designed in a way to be attached to other pin type distractors besides a Caspar-type pin distractor. It could also be designed to be attached directly to a threaded bone screw. Different sized distractor arms or offset measurements can be made for pin distractors so that this clamp can also be used in the anterior thoracic and lumbar spine.

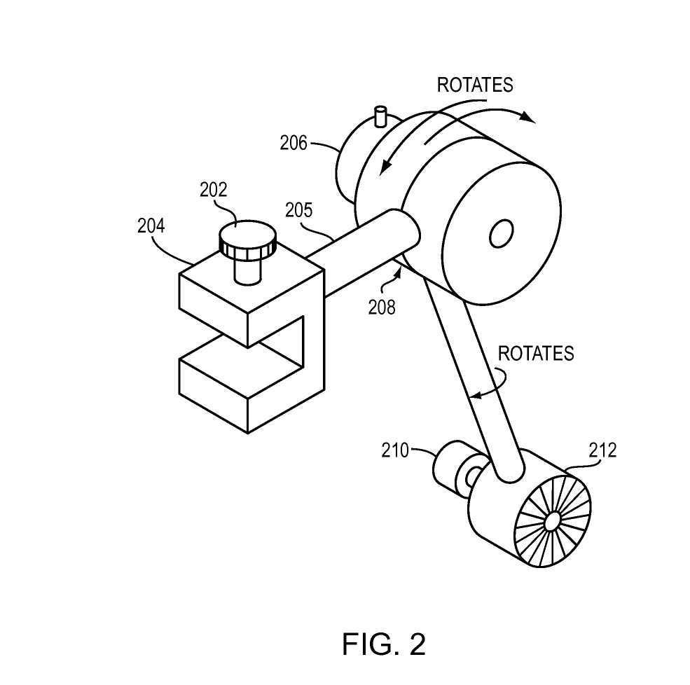

Turning now to FIG. 2, which illustrates one embodiment of the anterior spine array clamp, attachment clamp 204 is designed to mate removably with and be fixed to the bar of a Caspar-type pin distractor. Tightening screw 202 is a threaded screw that can be tightened to lock the attachment clamp 204 at the proximal end of the anterior spine array clamp to the distractor bar. Arm 205 is a rigid arm that connects attachment clamp 204 to rotational connector 206. Rotational connector 206 allows rotation about an axis normal to the long axis of arm 205. Rotational connector 206 can be fixed at a desired angle of rotation by tightening a screw that locks the rotating mechanism. Arm 208 is a second arm that connects rotational connector 206 and array mating surface 212 at the distal end of the anterior spine array clamp. Either of arm 206 and arm 208 can be designed to allow relative rotation of the structures at opposite ends of each arm about the long axis of each arm. Such rotation, if permitted, can be controlled by providing a screw that fixes the rotational relationship of each arm to the structures at either end of that arm. Tightening screw 210 is provided to allow a navigation array to be attached to the anterior spine array clamp at array mating surface 212. The relative orientation of the navigation array can be rotated about the axis of the tightening screw 212 until it is locked in place.

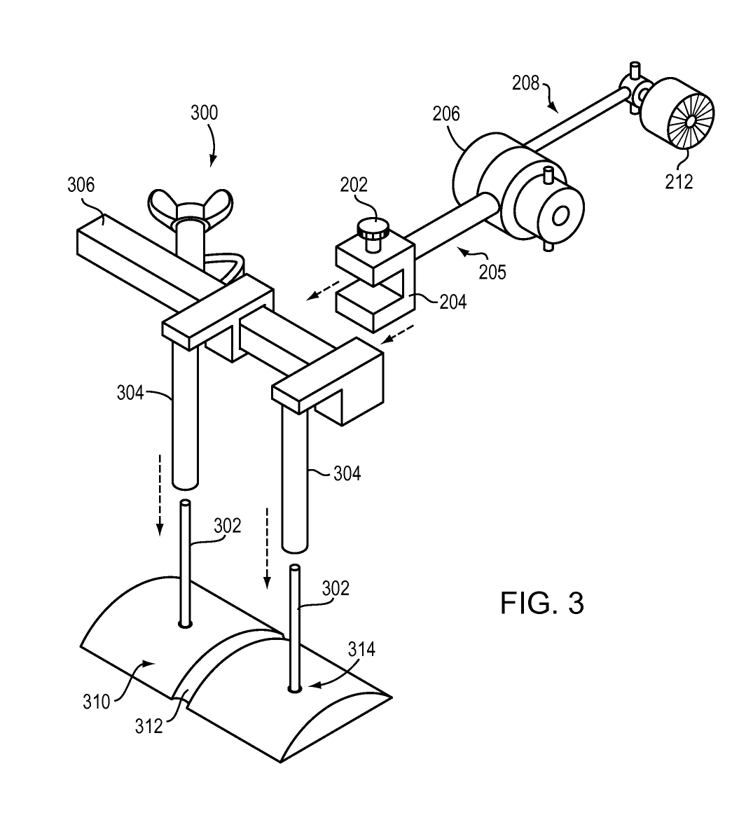

The method of use of the anterior spine array clamp is seen by referring to FIG. 3. In order to fix a navigation array to the anterior spine the surgeon installs a structure that when fully installed represents a fixed location relative to an intended surgical site in a body. In one embodiment, the structure can be a Caspar-type pin distractor. The surgeon inserts threaded fixation pins 302 (“caspar type pins”) to the vertebral body. One pin is threadedly inserted into one vertebra 310 and another pin is threadedly inserted into an adjacent vertebra 314. Disc 312 is a spinal disc situated between the two vertebra 310 and 314. The legs 304 of the pin distractor 300 are placed on the pins 302 (as indicated by the dotted arrows) and the pin distractor 300 is locked in its final position. Bar 306 of the pin distractor serves to space the legs 30 and the pins, and also provides a support location for application of the attachment clamp 204 of the anterior spine array clamp. The anterior spine array clamp is attached to the pin distractor bar 306 with tightening screw 202. The navigation array is attached to the mating surface 212, is adjusted as to its position, and locked in place with tightening screw 210. The arm 208 is adjusted by rotating it, by adjusting the relative angular relationships between arms 205 and 208, and /or by rotating about arm 205. Once the proper location of the navigation system is determined, based on the criterion that the navigation system is able to read the array, the components of the anterior spine array clamp are locked in place by tightening the respective tightening screws for a time period during the surgical operation.

In one embodiment, the anterior spine array clamp apparatus is constructed of material that can be sterilized repeatedly. In preferred embodiments, construction using titanium, aluminum and steel, alone or in combination, are viable options. In some other embodiments, other materials of construction may be used, such as engineering plastics that are suitable for use in a surgical environment, including Acrylonitrile butadiene styrene (ABS), Polycarbonates (PC), Polyamides (PA), Polybutylene terephthalate (PBT), Polyethylene terephthalate (PET), Polyphenylene oxide (PPO), Polysulphone (PSU), Polyetherketone (PEK), Polyetheretherketone (PEEK), Polyimides, Polyphenylene sulfide (PPS), and Polyoxymethylene plastic (POM).

Any patent, patent application, or publication identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. An anterior spine array clamp, comprising:
an attachment clamp configured to mate removably with and configured to be fixed to a fixed location relative to an intended surgical site in a body of a patient, said attachment clamp representing a proximal end of said anterior spine array clamp;
a first rigid arm having a first end adjustably attached to said attachment clamp and having a second end, and having a rotation axis disposed along the length of said first arm;
a rotational connector adjustably attached to said second end of said first arm;
a second rigid arm having a first end adjustably attached to said rotational connector and having a second end, and having a rotation axis disposed along the length of said second arm; and
an array mating surface adjustably attached to said second end of said second arm, said array mating surface representing a distal end of said anterior spine array clamp, said array mating surface configured to allow a navigation array to be attached thereto;
each of said attachment clamp, said first rigid arm, said rotational connector, said second rigid arm and said array mating surface provided with tightening screws configured to be operated manually, and each of said attachment clamp, said first rigid arm, said rotational connector, said second rigid arm and said array mating surface configured to be locked in place by tightening a respective tightening screw for a time period during a surgical operation.

2. The anterior spine array clamp of claim 1, wherein said fixed location relative to an intended surgical site in a body of a patient is a location in or on a mechanical device attached to said patient.

3. The anterior spine array clamp of claim 2, wherein said fixed location relative to an intended surgical site in a body of a patient is a location in or on a Caspar-type pin distractor attached to said patient.

4. The anterior spine array clamp of claim 1, constructed from a material that can be sterilized repeatedly.

5. The anterior spine array clamp of claim 4, wherein said material is selected from the group consisting of titanium, aluminum and steel.

6. A method of using an anterior spine array clamp, comprising the steps of:
providing an anterior spine array clamp comprising:
an attachment clamp configured to mate removably with and configured to be fixed to a fixed location relative to an intended surgical site in a body of a patient, said attachment clamp representing a proximal end of said anterior spine array clamp;
a first rigid arm having a first end adjustably attached to said attachment clamp and having a second end, and having a rotation axis disposed along the length of said first arm;
a rotational connector adjustably attached to said second end of said first arm;
a second rigid arm having a first end adjustably attached to said rotational connector and having a second end, and having a rotation axis disposed along the length of said second arm; and
an array mating surface adjustably attached to said second end of said second arm, said array mating surface representing a distal end of said anterior spine array clamp, said array mating surface configured to allow a navigation array to be attached thereto;
each of said attachment clamp, said first rigid arm, said rotational connector, said second rigid arm and said array mating surface provided with tightening screws configured to be operated manually, and each of said attachment clamp, said first rigid arm, said rotational connector, said second rigid arm and said array mating surface configured to be locked in place by tightening a respective tightening screw for a time period during a surgical operation;
attaching a structure to a location proximal to said intended surgical site in a body of a patient;
removably attaching said attachment clamp of said anterior spine array clamp to said structure; and
adjusting and locking in place said attachment clamp, said first rigid arm, said rotational connector, said second rigid arm and said array mating surface in relative positions.

7. The method of using an anterior spine array clamp of claim 6, further comprising the step of:
attaching a navigation array to said array mating surface of said anterior spine array clamp.

8. The method of using an anterior spine array clamp of claim 7, further comprising the step of:
adjusting a position of any of said attachment clamp, said first rigid arm, said rotational connector, said second rigid arm, said array mating surface and said navigation array relative to any other of said attachment clamp, said first rigid arm, said rotational connector, said second rigid arm, said array mating surface and said navigation array.
